(19) United States
(12) Patent Application Publication
(10) Pub. No.: US 2011/0313260 A1
(43) Pub. Date: Dec. 22, 2011

(54) DIAPER-TYPE VITAL SIGN MEASURING APPARATUS

(75) Inventors: Ji Wook JEONG, Daejeon (KR); Yong Won Jang, Daejeon (KR); Sung Yeup Hong, Daejeon (KR); Soo Yeul Lee, Daejeon (KR); Seung Hwan Kim, Daejeon (KR)

(73) Assignees: EULJI UNIVERSITY INDUSTRY COOPERATION, Daejeon (KR); ELECTRONICS AND TELECOMMUNICATIONS RESEARCH INSTITUTE, Daejeon (KR)

(21) Appl. No.: 13/161,677

(22) Filed: Jun. 16, 2011

(30) Foreign Application Priority Data
Jun. 18, 2010 (KR) 10-2010-0057972

(51) Int. Cl. A61B 5/00; A61B 5/08

(52) U.S. Cl. 600/301; 600/534

(57) ABSTRACT

Provided is a vital sign measuring apparatus capable of measuring vital signs such as a temperature of a human body, breathing, acceleration, oxygen saturation, voice, etc. to analyze and display a health index. The diaper-type vital sign measuring apparatus includes a vital sign measuring part for detecting variation in abdominal circumference according to breathing of a wearer to measure a vital sign of the breathing of the wearer, a vital sign analysis part for analyzing the vital sign measured by the vital sign measuring part to calculate a health index of the wearer, and a display part for displaying the health index calculated by the vital sign analysis part.

### CROSS-REFERENCE TO RELATED APPLICATION

This application claims the benefit of Korean Patent Application No. 10-2010-0057972, filed Jun. 18, 2010, the disclosure of which is hereby incorporated herein by reference in its entirety.

### BACKGROUND

1. Field of the Invention

The present invention relates to a diaper-type vital sign measuring apparatus, and more particularly, to a vital sign measuring apparatus capable of measuring vital signs such as a temperature of a human body, breathing, acceleration, oxygen saturation, voice, etc. to analyze and display a health index.

2. Discussion of Related Art

In recent times, sudden infant death syndrome (SIDS) marked by the sudden death of a healthy infant less than one year with no signs or causes has become an issue. This syndrome is known to most frequently occur in one- to four-month old infants. However, its causes have still not been clearly investigated, and infant mortality is increasing.

Therefore, in order to prevent the sudden death of infants, a necessity to develop a compact module for alarming a guardian of breathing or heartbeat stoppage so that first aid may be administered is increasing.

Meanwhile, diapers are generally used for defecation management and clean nurture of infants. In recent times, various functional diapers and disposable diapers, which have excellent moisture absorption, can detect and notify when the infant has defecated, and are improved in wear sensation, have been developed and entered the market.

In addition, diapers having various added functions of checking physical conditions of infants such as a change in temperature, movement, position, posture, etc., in addition to the defecation management of infants as basic function of the diapers are being developed.

However, it is difficult for the conventional diapers to prevent the sudden death of infants as described above.

In the case of sudden death, an emergency occurs at the time that breathing finally stops. The longer the time for which oxygen supply is stopped by breathing stoppage, the higher the incidence of difficulties and mortality. Therefore, in order to prevent the sudden death of infants, it is essential to monitor breathing of the infants.

However, since the functions of the conventional diapers are limited to check defecation state of infants or check simple health indices such as a body temperature, etc., the conventional diapers are not appropriate to monitor the breathing of infants to prevent the sudden death of infants.

### SUMMARY OF THE INVENTION

The present invention, therefore, solves the aforementioned problems associated with conventional devices by providing a diaper-type vital sign measuring apparatus capable of monitoring the breathing of infants to rapidly give first aid when dyspnea occurs.

According to an aspect of the present invention, a diaper-type vital sign measuring apparatus includes a vital sign measuring part for detecting a variation in abdomen circumference depending on breathing of a wearer to measure a vital sign of the breathing of the wearer; a vital sign analysis part for analyzing the vital sign measured by the vital sign measuring part to calculate a health index of the wearer; and a display part for displaying the health index calculated by the vital sign analysis part.

### BRIEF DESCRIPTION OF THE DRAWINGS

The above and other features of the present invention will be described in reference to certain exemplary embodiments thereof with reference to the attached drawings in which:

FIG. 1 is a block diagram showing constitution of a diaper-type vital sign measuring apparatus in accordance with an exemplary embodiment of the present invention;

FIG. 2A is a perspective view of a diaper-type vital sign measuring apparatus in accordance with a first exemplary embodiment of the present invention;

FIG. 2B is a perspective view of a diaper-type vital sign measuring apparatus in accordance with a second exemplary embodiment of the present invention; and

FIG. 3 is a graph showing a vital sign measured by the diaper-type vital sign measuring apparatus in accordance with an exemplary embodiment of the present invention.

### DETAILED DESCRIPTION OF PREFERRED EMBODIMENTS

The present invention will now be described more fully hereinafter with reference to the accompanying drawings, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art. Like numbers refer to like elements throughout the specification.

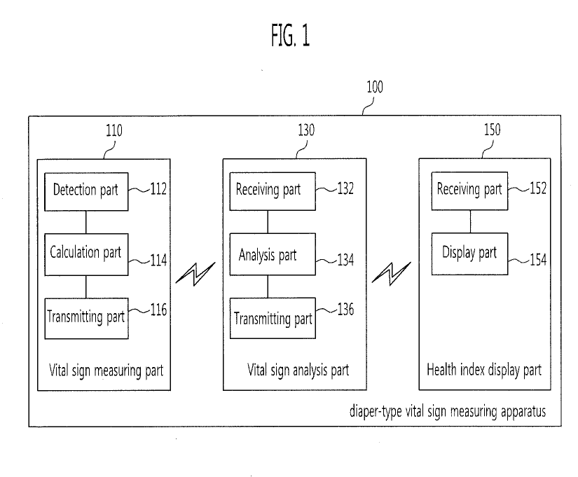

FIG. 1 is a block diagram showing constitution of a diaper-type vital sign measuring apparatus in accordance with an exemplary embodiment of the present invention.

As shown, a diaper-type vital sign measuring apparatus 100 in accordance with an exemplary embodiment of the present invention includes a vital sign measuring part 110 for measuring a vital sign of a wearer, a vital sign analysis part 130 for analyzing the vital sign measured by the vital sign measuring part 110 to calculate a health index, and a health index display part 150 for displaying the health index calculated by the vital sign analysis part 130.

The vital sign measuring part 110 includes a detection part 112 for measuring a vital sign of a wearer to convert the vital sign into an electrical signal, a calculation part 114 for collecting the signal output from the detection part 112 to AD-convert and filter the signal, and a transmitting part 116 for transmitting the signal output from the calculation part 114 to the vital sign analysis part 130. While not shown, the vital sign measuring part 110 may further include a storage part for storing the vital sign.

Here, the detection part 112 measures a vital sign of a wearer using a resistive conductive wire or various sensors, and converts the measured vital sign into an electrical signal.

For example, when the resistive conductive wire is used, since a resistance value of the resistive conductive wire varies depending on a length, it is possible to detect a variation in resistance according to a variation in length of the resistive conductive wire to measure a vital sign of the breathing of the wearer, i.e., a breathing sign.

Such a resistive conductive wire may be mounted on the front surface of a diaper in a circumferential direction thereof, and thus, the length of the resistive conductive wire varies to correspond to a variation in abdominal circumference according to the breathing of the wearer.

For another example, the detection part 112 may further include a temperature sensor, a pressure sensor, an acceleration sensor, an oxygen saturation sensor, a voice sensor, or a humidity sensor so that various vital signs of the wearer can be measured using the various sensors.

When the temperature sensor is used, a temperature sign can be measured to indicate a vital sign of the body temperature of the wearer. When the pressure sensor is used, a pressure sign can be measured to indicate a vital sign of a posture, a variation in posture or breathing of the wearer. When the acceleration sensor is used, a movement sign or an acceleration sign can be measured to indicate a vital sign of a variation in movement of the wearer. When the oxygen saturation sensor is used, an oxygen saturation sign can be measured to indicate a vital sign of oxygen saturation of the wearer. When the voice sensor is used, a voice sign can be measured as a vital sign to check whether the wearer is crying or not. In addition, when the humidity sensor is used, a humidity sign for representing a variation in humidity depending on defecation of the wearer can be measured.

When the vital signs are measured using the sensors, the sensors may be attached to appropriate positions selected such that the corresponding vital signs can be most effectively measured.

The calculating part 114 may include a microprocessor or a digital filter. The digital filter may include a digital low-pass filter or a digital high-pass filter, and the digital low-pass filter may have a cutoff frequency of about 1 Hz.

In addition, the calculation part 114 may include a voltage distribution circuit, and the voltage distribution circuit may include a variable resistor.

Further, the calculation part 114 may include an AD converter or a signal converter for converting a digital signal according to communication standards.

The transmitting part 116 may transmit a vital sign converted into a digital signal according to communication standards using a wired communication method or a wireless communication method. The wired communication method may be a serial communication method, and the wireless communication method may be a Bluetooth communication method, a Zigbee communication method, or a mobile communication method.

The vital sign analysis part 130 includes a receiving part 132 for receiving a converted vital sign from the vital sign measuring part 110, an analysis part 134 for analyzing the received vital sign to calculate a health index, and a transmitting part 136 for transmitting the calculated health index to the health index display part 150. Although not shown, the vital sign analysis part 130 may further include a storage part for storing the received vital sign or the calculated health index.

The receiving part 132 may be a wired receiving part or a wireless receiving part. The wired receiving part may be a serial communication receiving part. The wireless receiving part may be a Bluetooth communication receiving part, a Zigbee communication receiving part, or a mobile communication receiving part.

The analysis part 134 analyzes the received vital sign to calculate a health index, and generates an emergency signal or a notification signal according to the calculated health index.

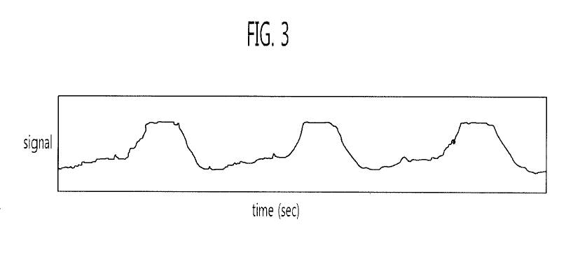

For example, the analysis part 134 may analyze the number of peaks, heights of peaks, and so on, of the breathing sign measured using the resistive conductive wire to calculate a health index of the number of breaths, a breathing strength or apnea/hypopnea of a wearer. A specific calculation method will be described below with reference to FIG. 3.

For example, the analysis part 134 may analyze various vital signs measured using the temperature sensor, the pressure sensor, the acceleration sensor, the oxygen saturation sensor, the voice sensor, or the humidity sensor, to calculate a health index.

That is, it is possible to analyze the temperature sign measured using the temperature sensor to calculate a variation in temperature of the wearer. In addition, it is possible to analyze a degree of disorder or an amplitude of the acceleration sign measured using the acceleration sensor to calculate activity of the wearer, or it is possible to analyze a change in direction of the acceleration sign to calculate movement of the wearer, for example, the inclination of the wearer's body. Further, it is possible to analyze the oxygen saturation sign measured using the oxygen saturation sensor to calculate the oxygen saturation of the wearer, or it is possible to analyze the humidity sign measured using the humidity sensor to calculate the defecation state of the wearer. Furthermore, it is possible to analyze the voice sign measured using the voice sensor to determine whether the wearer is crying.

As described above, the analysis part 134 may analyze the vital sign to recognize physical conditions of the wearer using the calculated health index and generate an emergency signal when an emergency occurs. For example, the analysis part 134 may generate the emergency signal for alarming of the emergency when it is determined that the breathing, the breathing strength, the oxygen saturation, etc., are not in a normal range or that the wearer is in an apnea/hypopnea state.

In addition, when the emergency occurs, for example, when the humidity of the diaper is increased by the defecation or when the infant cries, it is possible to generate a notification signal.

Further, the analysis part 134 may generate a guide signal including guide information when an emergency or notification situation occurs. For example, when the emergency situation occurs, the analysis part 134 may generate a guide signal including emergency treatment information with an emergency signal for notifying of the occurrence of the emergency situation. Furthermore, when the notification situation occurs, the analysis part 134 may generate a guide signal to output music or vibrations for psychological stability of the wearer with the notification signal for notifying of the occurrence of the notification situation.

The health index display part 150 includes a receiving part 152 for receiving a health index from the vital sign analysis part 130, and a display part 154 for displaying the received health index.

The receiving part 152 receives the health index, the emergency signal, the notification signal, the guide signal, etc., calculated by the analysis part 134 of the vital sign analysis part 130.

The display part 154 may be mounted on a diaper or constituted by a separate terminal to display a health index of the wearer or display an emergency signal, a notification signal or a guide signal.

For example, when the display part 154 is mounted on the diaper, the display part 154 may be implemented by a speaker, an LED, a vibrator, etc. In this case, the display part 154 generates an alarm sound using the speaker or displays a light using the LED in response to the reception of the emergency or notification signal. In addition, according to the received guide signal, music or vibrations may be output to psychologically stabilize the wearer.

For another example, when the display part 154 is constituted by a separate terminal, the display part 154 may display a popup window on a monitor to show the received emergency signal, notification signal or guide signal.

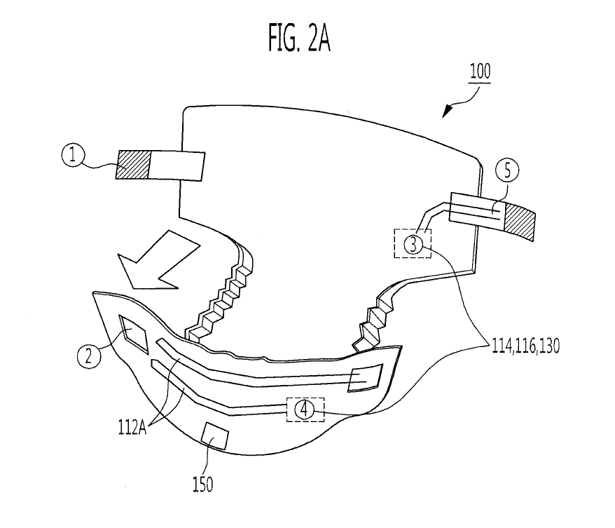

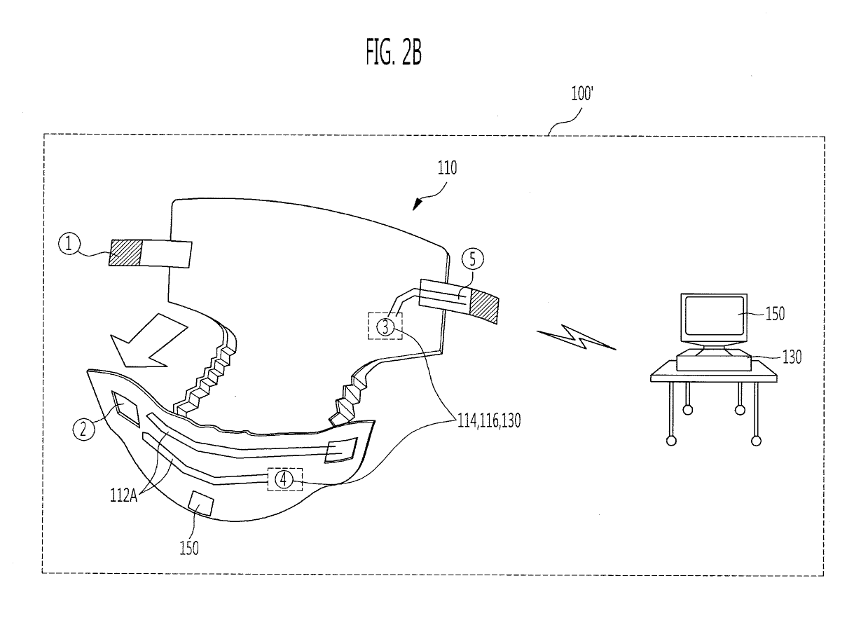

FIG. 2A is a perspective view of a diaper-type vital sign measuring apparatus in accordance with a first exemplary embodiment of the present invention.

As shown, a diaper-type vital sign measuring apparatus 100 in accordance with a first exemplary embodiment of the present invention includes a measuring part 110 mounted on a diaper, a vital sign analysis part 130, and a health index display part 150. In particular, in this embodiment, the case in which a detection part 112 of the vital sign measuring part 110 includes at least one resistive conductive wire 112A will be described.

Here, the resistive conductive wire 112A is mounted on a portion of the diaper in which a dimension varies depending on the breathing in consideration of anatomic characteristics of the wearer. For example, the wire may be attached to a front surface of the diaper in a circumferential direction thereof, and in particular, to a position corresponding to an upper abdomen of the wearer. Here, the resistive conductive wire 112A may be mounted around the upper abdomen to both side regions in a lateral strip shape, or mounted around the chest near the diaphragm to both side regions in a lateral strip shape.

In addition, the resistive conductive wire 112A may be formed in a single wire shape or a woven wire shape. In particular, the resistive conductive wire 112A may include a conductive fiber and an elastic thread, and may have a shape twisted with the conductive fiber and elastic thread, a shape in which conductive fibers are woven around the elastic thread, or a shape in which conductive fibers are woven around the elastic thread in two layers in opposite directions. Further, the resistive conductive wire 112A may include a carbon fiber or a metal wire.

Furthermore, the resistive conductive wire 112A may be formed as a part of the diaper or may be attached to the diaper using an attachment material.

The vital sign measured by the resistive conductive wire 112A is processed by the calculation part 114, the transmitting part 116 and the vital sign analysis part 130, which may be connected to a front surface \textcircled{4} of the diaper to be mounted thereon, or a rear surface \textcircled{3} of the diaper, i.e., a rear side of the wearer's waist, to be mounted thereon by the resistive conductive wire 112A.

Here, when the calculation part 114, the transmitting part 116 and the vital sign analysis part 130 are mounted on the rear surface \textcircled{3} of the diaper, the vital sign measured by the detection part 112 is transmitted to the calculation part 114 through wired or wireless communication.

For example, the resistive conductive wire 112A may be connected to the calculation part 114 mounted on the rear surface by detachably attaching the resistive conductive wire 112A and the calculation part 114 using attachment bands \textcircled{1} and \textcircled{2} and mounting a connection wire \textcircled{5} in the band \textcircled{1}. In addition, the resistive conductive wire 112A may be connected to the calculation part 114 by mounting connection parts on both ends of the connection parts such as buttons of the connection wire \textcircled{5} and the resistive conductive wire 112A and attaching the connection part of both ends. Here, the connection wire \textcircled{5} may be a copper wire or other resistive conductive wire.

The health index calculated by the vital sign analysis part 130 is displayed through the display part 150. In this embodiment, the display part 150 is mounted on a portion of the diaper, in particular, a front surface of the diaper. Of course, the display part 150 may be mounted on a position that can be easily observed by a guardian.

Here, the display part 150 may be an LED or a speaker. For example, the LED may be set to emit light in a specific circumstance to notify of a health index, an emergency situation, a notification situation, and so on, of the wearer. In addition, the speaker may be set to generate an alarm sound according to the health index, the emergency situation, or the notification situation of the wearer.

While attachment of sensors is not shown in the drawings, as described above, the sensors may be attached to positions appropriate to measure vital signs to measure various vital signs.

FIG. 2B is a perspective view of a diaper-type vital sign measuring apparatus in accordance with a second exemplary embodiment of the present invention.

As shown, a vital sign measuring apparatus 100′ in accordance with a second exemplary embodiment of the present invention includes a vital sign measuring part 110 mounted on a diaper, and a vital sign analysis part 130 and a health index display part 150, which are constituted as a separate terminal.

Here, the separate terminal may be a mobile phone or a computer. A guardian can remotely manage the health index of the wearer using the terminal, and receive an emergency signal or a notification signal in real time when an emergency situation or a notification situation occurs.

Other constitutions are the same as the first embodiment.

FIG. 3 is a graph showing a vital sign measured by the diaper-type vital sign measuring apparatus in accordance with an exemplary embodiment of the present invention.

As described above, the diaper-type vital sign measuring apparatus in accordance with an exemplary embodiment of the present invention can measure a breathing sign according to the breathing of the wearer using the resistive conductive wire. The graph shows breathing signs, in which an x-axis represents time and a y-axis represents voltage of the breathing sign.

The vital sign analysis part 130 analyzes the measured breathing sign to calculate a health index.

For example, since the length of the resistive conductive wire 112A varies to form a peak of the breathing sign whenever the wearer breathes, it is possible to analyze the number of peaks of the breathing signs in a predetermined time to calculate the number of breaths of the wearer at that moment.

In addition, since the length of the resistive conductive wire 112A varies to vary the height of the peak in proportion to the breathing depth of the wearer, the breathing strength can be calculated by measuring the height of the peak. Otherwise, it is possible to analyze tendency of the calculated breathing strength to infer variation in breathing strength.

Further, it is possible to analyze the number of the calculated breaths and the breathing strength to determine whether the wearer is in an apnea/hypopnea state.

While the diaper-type vital sign measuring apparatus in accordance with the present invention capable of managing a health index of infants and detecting an emergency situation such as apnea/hypopnea in real time has been described, this is provided for the convenience of description only, and the present invention is not limited thereto. The diaper-type vital sign measuring apparatus in accordance with an exemplary embodiment of the present invention may be applied to the old and the infirm, patients, and so on, which cannot move about freely or cannot easily treat emergency situations, in addition to infants.

As can be seen from the foregoing, it is possible to detect variation in abdominal circumference according to breathing of a wearer to measure a vital sign of the breathing in real time, and it is possible to analyze the measure of vital signs to calculate health indices such as the number of breaths, breathing strength, apnea/hypopnea of a wearer, etc. In addition, it is possible to transmit an emergency alarm to a guardian in a wired/wireless communication system on the basis of the calculated health index upon occurrence of the emergency. Therefore, it is possible to give first aid when the emergency such as breathing disability of the wearer occurs.

In particular, it is possible to measure a vital sign of a wearer using a resistive conductive wire having a length varied in response to a variation in abdominal circumference depending on the breathing of the wearer, stably measuring the number of breaths of the wearer. Moreover, since the resistive conductive wire has good electrical characteristics to enable precise measurement of an electrical signal of a living body and easy combination with a diaper-type mechanism, an effective vital sign measuring apparatus can be manufactured at a low cost.

In addition, sensors such as a temperature sensor, an oxygen saturation sensor, an acceleration sensor, a pressure sensor, etc., may be additionally mounted on the diaper-type vital sign measuring apparatus to measure essential vital signs such as a temperature sign, an acceleration sign, oxygen saturation, a voice sign, etc., in addition to the breathing of the wearer. Therefore, the physical condition of the wearer can be effectively monitored.

Although the present invention has been described with reference to certain exemplary embodiments thereof, it will be understood by those skilled in the art that a variety of modifications and variations may be made to the present invention without departing from the spirit or scope of the present invention defined in the appended claims, and their equivalents.

What is claimed is:

1. A diaper-type vital sign measuring apparatus comprising:
a vital sign measuring part for detecting variation in abdomen circumference according to breathing of a wearer to measure a vital sign of the breathing of the wearer;
a vital sign analysis part for analyzing the vital sign measured by the vital sign measuring part to calculate a health index of the wearer; and
a display part for displaying the health index calculated by the vital sign analysis part.

2. The diaper-type vital sign measuring apparatus according to claim 1, wherein the vital sign measuring part comprises a resistive conductive wire mounted on a front surface of a diaper in a circumferential direction and having a length that varies depending on a variation in abdomen circumference when the wearer breathes.

3. The diaper-type vital sign measuring apparatus according to claim 2, wherein the vital sign measuring part detects a variation in resistance depending on a variation in length of the resistive conductive wire to measure a vital sign of the breathing of the wearer.

4. The diaper-type vital sign measuring apparatus according to claim 2, wherein the vital sign analysis part analyzes the vital sign measured using the resistive conductive wire to calculate a health index of the number of breaths, breathing strength, or apnea/hypopnea of the wearer.

5. The diaper-type vital sign measuring apparatus according to claim 1, wherein the vital sign measuring part comprises:
a detection part for measuring a vital sign of the wearer to convert the vital sign into an electrical signal;
a calculation part for collecting a signal output from the detection part to analog-digital convert and filter the signal; and
a transmitting part for transmitting the signal output from the calculation part to the vital sign analysis part.

6. The diaper-type vital sign measuring apparatus according to claim 1, wherein the vital sign measuring part comprises at leas one of a temperature sensor, a pressure sensor, an acceleration sensor, an oxygen saturation sensor, a voice sensor and a humidity sensor, which are mounted on the diaper.

7. The diaper-type vital sign measuring apparatus according to claim 6, wherein the vital sign analysis part analyzes the vital sign measured by the vital sign measuring part to calculate a health index of a body tilt, activity, temperature, oxygen saturation, defecation, or crying of the wearer.

8. The diaper-type vital sign measuring apparatus according to claim 1, wherein the vital sign analysis part is mounted on the diaper or constituted by a separate terminal.

9. The diaper-type vital sign measuring apparatus according to claim 1, wherein the display part is mounted on the diaper or constituted by a separate terminal.

10. The diaper-type vital sign measuring apparatus according to claim 9, wherein the display part comprises at least one of an LED, a speaker, and a vibrator, which are mounted on the diaper.
